(19) United States
(12) Patent Application Publication
(10) Pub. No.: US 2024/0261216 A1
(43) Pub. Date: Aug. 8, 2024

(54) AGENT FOR DYEING KERATIN MATERIAL, IN PARTICULAR HUMAN HAIR, CONTAINING AMINOSILICONES, PIGMENTS AND THE SALTS OF DIVALENT CATIONS

(71) Applicant: HENKEL AG & CO. KGAA, Düsseldorf (DE)

(72) Inventors: Constanze KRUCK, Grevenbroich (DE); Gabriele WESER, Essen (DE); Marcus CLAAS, Hilden (DE); Marie HENSEL, Köln (DE); Ranju Prasad MANDAL, Düsseldorf (DE); Imme BREUER, Düsseldorf (DE)

(21) Appl. No.: 18/290,546

(22) PCT Filed: Dec. 7, 2022
(86) PCT No.: PCT/EP2022/084744

§ 371 Date: Nov. 14, 2023

(30) Foreign Application Priority Data
Jan. 26, 2022 (DE) 10 2022 200 862.6

(51) Int. Cl. A61K 8/898; A61K 8/20; A61K 8/39; A61K 8/86; A61Q 5/06

(52) U.S. Cl. CPC A61K 8/898; A61K 8/20; A61K 8/39; A61K 8/86; A61Q 5/065; A61K 2800/43

(57) ABSTRACT

The present invention relates to an agent for dyeing keratin material, in particular human hair, containing
(a1) at least one salt of a divalent cation,
(a2) at least one pigment, and
(a3) at least one amino-functionalized silicone polymer.

The present application relates to an agent for dyeing keratin material, in particular human hair, which contains at least one salt of a divalent cation (a1), at least one pigment (a2), and at least one amino-functionalized silicone polymer (a3).

This application further relates to a method for dyeing keratin material, in particular human hair, the agent described above being applied to the keratin material, or to the human hair, and being optionally rinsed out again after an exposure time of 30 seconds to 45 minutes.

Changing the shape and color of keratin material, in particular human hair, represents an important field of modern cosmetics. To change the hair color, the skilled artisan is familiar with a variety of dyeing systems depending on the dyeing requirements. Oxidation dyeing agents are typically used for permanent, intense dyeing with good fastness properties and good gray coverage. Such dyeing agents contain oxidation dye precursors, what are known as developer components and coupler components, which together form the actual dyes under the influence of oxidizing agents such as, for example, hydrogen peroxide. Oxidation dyes are characterized by very long-lasting color results.

When using direct dyes, dyes which are already formed diffuse out of the dyeing agent into the hair fiber. Compared to oxidative hair coloring, the colorings obtained with direct dyes are not as durable and wash out more quickly. Colors with direct dyes usually remain on the hair for a period of between 5 and 20 hair washes.

The use of color pigments for brief changes in color on the hair and/or the skin is known. Color pigments are generally understood to mean insoluble dyeing substances. These are present undissolved in the form of small particles in the dyeing formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting dyeing, the use of oxidative dyeing agents has hitherto been the only option. However, despite multiple optimization attempts, an unpleasant ammonia odor or amine odor cannot be completely avoided in oxidative hair dyeing. The hair damage that remains associated with the use of the oxidative dyeing agents also has a disadvantageous effect on the hair of the user. A continuing challenge is therefore the search for alternative, high-performance dyes and dyeing processes. Recently, there has been a particular focus on dyeing systems based on pigments.

The object of the present invention is that of providing a dyeing agent which makes it possible to fix pigments to the hair in an extremely durable manner. When using the agent in a dyeing method, particularly intensive dyeing results with good wash fastness, good rubbing fastness, good leveling capacity and a particularly uniform color result should be achieved. The improvement in the wash fastness of the colorings obtained with pigments is very particularly the focus in this case.

Surprisingly, it has been found that the aforementioned object can be achieved comprehensively manner when keratin material, in particular human hair, is colored with an agent which contains at least one salt of a divalent cation (a1), at least one pigment (a2), and at least one amino-functionalized silicone polymer (a3).

A first subject matter of the present invention is an agent for dyeing keratin material, in particular human hair, containing:
(a1) at least one salt of a divalent cation, and
(a2) at least one pigment, and
(a3) at least one amino-functionalized silicone polymer.

Within the context of the work leading to this invention, it was found that it was possible to obtain especially good color results when the pigment or pigments, in admixture with at least one salt of a divalent cation, and at least one amino-silicone, were applied to the keratin material, in particular human hair. In this way, the wash fastness of the colored hair could be improved particularly strongly.

### Keratin Material

Keratin material is understood to mean hair, skin, and nails (such as, for example, fingernails and/or toenails). Furthermore, wool, furs and feathers also fall under the definition of the keratin material.

Keratin material is preferably understood to be human hair, human skin and human nails, in particular fingernails and toenails. Keratin material is very particularly preferably understood to mean human hair.

### Dyeing Agent

Within the scope of this invention, the term “dyeing agent” is used for a coloring of the keratin material, in particular hair, brought about by use of pigments. With this coloring, the pigments as coloring compounds are deposited in a particularly homogeneous and uniform film on the surface of the keratin material.

According to the invention, the dyeing agent represents a ready-to-use agent. This ready-to-use agent can, for example, be filled into a container and applied to the keratin material in this form without further dilution, mixing or other method steps. For reasons of storage stability, however, it has been found to be very particularly preferred for the ready-to-use cosmetic agent to be prepared by the hairdresser or user only shortly before use. To prepare the ready-to-use agent, for example, the salt of the divalent cation (a1) and pigment (a2) can be mixed or predispersed with one or more additional agents, one of these additional agents containing at least one amino-functionalized silicone polymer (a3). However, it is also just as conceivable for the ready-to-use agent to be produced by mixing at least three different agents, one of these agents containing at least one salt of a divalent cation (a1), an additional agent containing at least one pigment (a2), and still another agent containing at least amino-functionalized silicone polymer (a3). The means can be mixed, for example, by being shaken and in this way ensures a very particularly uniform distribution of the dispersed pigments.

The agent or the ready-to-use agent contains the components (a1), (a2) and (a3) essential to the invention preferably in a cosmetic carrier.

In other words, a first subject matter of the present invention is an agent for dyeing keratinous material, in particular human hair, containing, in a cosmetic carrier:
(a1) at least one salt of a divalent cation, and
(a2) at least one pigment, and
(a3) at least one amino-functionalized silicone polymer.

### Salts of Divalent Cations (a1)

As a first essential component, the agent according to the invention contains at least one salt of a divalent cation (a1).

In the context of this invention, divalent cations are to be understood as meaning atoms and molecules, in particular atoms, having two positive charges. These doubly positively charged cations are neutralized by the presence of a corresponding number of anions.

Salts refer to chemical compounds which are formed from the positively charged cations and negatively charged anions, wherein there are ionic bonds between cation and anion.

Highly suitable divalent cations are, for example, the doubly positively charged alkaline earth cations Mg²⁺, Ca²⁺ and Ba²⁺, in particular Mg²⁺ (doubly positively charged magnesium) and Ca²⁺ (doubly positively charged calcium).

The anions neutralizing these positive charges can be inorganic or organic.

Particularly well-suited salts are the chlorides, bromides, hydroxides, citrates, salicylates, phosphates, sulfates, tartrates, lactates, malates, succinates, benzoates, fumarates, maleic salts and acetates of divalent cations, in particular the aforementioned salts of magnesium and/or calcium.

It has therefore been found to be particularly advantageous for achieving the object of the invention if the agent according to the invention contains at least one salt of a divalent calcium cation and/or magnesium cation (a1), which is preferably selected from the group consisting of calcium chloride, magnesium chloride, calcium bromide, magnesium bromide, calcium hydroxide, magnesium hydroxide, calcium citrate, magnesium citrate, calcium salicylate, magnesium salicylate, calcium phosphate, magnesium phosphate, calcium sulfate, magnesium sulfate, calcium tartrate, magnesium tartrate, calcium lactate, magnesium lactate, calcium malate, magnesium malate, calcium succinate, magnesium succinate, calcium benzoate, magnesium benzoate, calcium fumarate, magnesium fumarate, calcium maleinate, magnesium maleinate, calcium acetate and magnesium acetate.

In the context of a particularly preferred embodiment, an agent according to the invention is therefore characterized in that it contains
(a1) at least one salt of a divalent calcium cation and/or magnesium cation, which is preferably selected from the group consisting of calcium chloride, magnesium chloride, calcium bromide, magnesium bromide, calcium hydroxide, magnesium hydroxide, calcium citrate, magnesium citrate, calcium salicylate, magnesium salicylate, calcium phosphate, magnesium phosphate, calcium sulfate, magnesium sulfate, calcium tartrate, magnesium tartrate, calcium lactate, magnesium lactate, calcium malate, magnesium malate, calcium succinate, magnesium succinate, calcium benzoate, magnesium benzoate, calcium fumarate, magnesium fumarate, calcium maleinate, magnesium maleinate, calcium acetate and magnesium acetate.

Explicitly very particularly preferred is an agent according to the invention which is characterized in that it contains
(a1) at least one salt of a divalent calcium cation and/or magnesium cation which is selected from the group consisting of calcium chloride and magnesium chloride.

Calcium chloride carries molecular formula CaCl₂ and can be purchased commercially from various suppliers either in anhydrous form or in the form of various hydrates. Calcium chloride carries the following CAS numbers:
10043-52-4 (anhydrous), molar mass=110.98 g/mol
10035-04-8 (dihydrate), molar mass=147.02 g/mol
25094-02-4 (tetrahydrate), molar mass=183.04 g/mol
7774-34-7 (hexahydrate), molar mass=219.08 g/mol

Magnesium chloride carries molecular formula MgCl₂ and can be purchased commercially from various suppliers either in anhydrous form or in the form of various hydrates, in particular in the form of its hexahydrate. Magnesium chloride carries the following CAS numbers:
7786-30-3 (anhydrous), molar mass equal to 95.21 g/mol
7791-18-6 (hexahydrate), molar mass equal to 203.3 g/mol

According to a further preferred embodiment, the agent according to the invention contains the salt(s) of divalent cations (a1) in specific quantity ranges. In this case, particularly strong improvement of the wash fastness of the dyed keratin material was observed when the agent according to the invention contained, based on the total weight of the agent, one or more salts of divalent cations (a1) in a total amount from 0.1 to 25 wt. %, preferably 0.5 to 20 wt. %, more preferably from 1.0 to 15 wt. %, still more preferably from 1.5 to 10 wt. %, and very particularly preferably from 2.5 to 7.5 wt. %.

In another preferred embodiment, an agent according to the invention is characterized in that it contains, based on the total weight of the agent, one or more salts of divalent cations (a1) in a total amount from 0.1 to 25 wt. %, preferably 0.5 to 20 wt. %, more preferably from 1.0 to 15 wt. %, still more preferably from 1.5 to 10 wt. %, and very particularly preferably from 2.5 to 7.5 wt. %.

In a very particularly preferred embodiment, an agent according to the invention is characterized in that it contains, based on the total weight of the agent, one or more salts from the group of calcium chloride and magnesium chloride (a1) in a total amount from 0.1 to 25 wt. %, preferably from 0.5 to 20 wt. %, more preferably from 1.0 to 15 wt. %, still more preferably from 1.5 to 10 wt. %, and very particularly preferably from 2.5 to 7.5 wt. %.

The basis for calculating the quantities of the salts (a1) in percent by weight is always the use of the anhydrous salt, for example the anhydrous calcium chloride or the anhydrous magnesium chloride.

### EXAMPLE

If an agent contains 10.0 g of calcium chloride in the form of its hexahydrate (molar mass=219.08 g/mol), this corresponds to an amount of substance of 0.0456 mol, which corresponds to a use amount of 5.065 g of anhydrous calcium chloride.

### Pigments (a2)

As the second essential component, the agent according to the invention contains at least one pigment (a2). Pigments within the meaning of the present invention are understood to mean dyeing compounds which have a solubility of less than 0.5 g/L, preferably of less than 0.1 g/L, even more preferably of less than 0.05 g/L, at 25° C. in water. The water solubility can be determined, for example, by means of the method described below: 0.5 g of the pigment is weighed into a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable dye pigments may be of inorganic and/or organic origin.

In a preferred embodiment, an agent according to the invention is characterized in that it contains at least one dying compound (a2) from the group consisting of inorganic and/or organic pigments.

Preferred color pigments are selected from synthetic or natural inorganic pigments. Inorganic color pigments of natural origin can be produced, for example, from chalk, ocher, umbra, green soil, burnt Sienna or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred color pigments are black iron oxide (CI 77499), yellow iron oxide (CI 77492), red and brown iron oxide (CI 77491), manganese violet (CI 77742), ultramarine (sodium aluminum sulphosilicates, CI 77007, Pigment Blue 29), chromium oxide hydrate (CI77289), Iron Blue (ferric ferrocyanide, CI77510) and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, primarily muscovite or phlogopite, is coated with a metal oxide.

As an alternative to natural mica, synthetic mica coated with one or more metal oxides(s) can also be used as a pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In another preferred embodiment, an agent according to the invention is characterized in that it contains at least one inorganic pigment (a2), which is preferably selected from the group of colored metal oxides, metal hydroxides, metal oxide hydrates, silicates, metal sulfides, complex metal cyanides, metal sulfates, bronze pigments and/or from mica-based colored pigments which are coated with at least one metal oxide and/or a metal oxychloride.

In another preferred embodiment, an agent according to the invention is characterized in that it contains at least one dyeing compound (a2) from the group consisting of pigments selected from mica-based colored pigments which are coated with one or more metal oxides from the group consisting of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, pigment blue 29), chromium oxide hydrate (CI 77289), chromium oxide (CI 77288), and/or iron blue (ferric ferrocyanide, CI 77510).

Examples of particularly suitable color pigments are commercially available, for example, under the trade names Rona®, Colorona®, Xirona®, Dichrona® and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES)

Additional particularly preferred color pigments with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

In another embodiment, the agent according to the invention contains at least one pigment (a2) from the group of platelet-type metal pigments.

Metallic pigments are understood to mean pigments which comprise at least one metal and/or at least one metal alloy. Any metal suitable for metallic luster pigments is suitable as the metal. Such metals are, inter alia, iron and steel and, for example, platinum, zinc, chromium and molybdenum. Preferred metals are aluminum, copper, silver and gold. Aluminum is very particularly preferred. The metallic pigments according to the invention can also contain mixtures of metals which may be contained, for example, in the form of the corresponding alloys in the pigment. Suitable alloys are, for example, aluminum bronzes and brass.

Metals within the meaning of the present invention are the corresponding elements mentioned in the present case with the oxidation state 0, i.e., metal oxides such as aluminum oxide, iron oxide, zinc oxide or copper oxide are explicitly not to be understood as metals.

In another very particularly preferred embodiment, an agent according to the invention is characterized in that it contains at least one pigment (a2) from the group of platelet-type metal pigments, which comprises at least one metal from the group of aluminum, copper, silver, gold, platinum, zinc, chromium, molybdenum and iron, particularly preferably aluminum.

In a further very particularly preferred embodiment, an agent according to the invention is characterized in that it contains:
(a2) at least one platelet-type metallic pigment comprising aluminum.

The metallic pigments according to the invention are platelet-type. A platelet is understood to mean a three-dimensional body which can be regularly or irregularly shaped and whose width and depth have greater dimensions than its thickness. The average dimensions apply in each case as measurement points for the measurement of width, depth and thickness. Alternatively, the platelet-type constituents of the metallic pigment are also referred to as substrate plates.

Incident light is reflected as directed from a mirror onto the surfaces of the platelet shape of these metallic pigments. Its optical effect is based on the orientation of its platelets parallel to the surface of the surrounding system, which produces the metallic effect for an observer.

The platelet-type metallic pigment which is used in the dyeing agent according to the invention is present in the form of a large number of substrate plates. For example, a platelet-type aluminum pigment is a pigment consisting of a large number of substrate plates which comprise aluminum or are composed at least in part of elemental aluminum.

The platelet-type metallic pigments according to the invention preferably have a content of elemental metal of at least 50 wt. %, preferably of at least 60 wt. %, more preferably of at least 70 wt. %, even more preferably of at least 80 wt. %, even more preferably of at least 90 wt. %, in each case in relation to the weight of metallic pigment. If the platelet-type metallic pigment is coated, the aforementioned weight data refer to the weight of the uncoated metallic pigment. Within the meaning of the invention, the aforementioned content of elemental metal is also understood to mean the portion of the respective metal present in an alloy.

In another embodiment, the agent according to the invention can also contain one or more pigments (a2) from the group consisting of organic pigments.

The organic pigments according to the invention are correspondingly insoluble organic dyes or color lakes which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

Particularly well suited organic pigments can for example include carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, an agent according to the invention is characterized in that it contains at least one organic pigment (a2), which is preferably selected from the group consisting of carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570 or CI 74260, orange pigments with the Color Index numbers CI 11725, CI 15510, CI 45370, CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color lake. The term lake within the meaning of the invention is understood to mean particles which comprise a layer of absorbed dyes, with the unit consisting of particles and dye being insoluble under the above-mentioned conditions. The particles may be, for example, inorganic substrates which may be aluminum, silica, calcium borosilicate, calcium aluminum borosilicate or aluminum.

For example, the alizarin color lake can be used as the color lake.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has a mean particle size D₅₀ from 1.0 to 50 μm, preferably from 5.0 to 45 μm, preferably from 10 to 40 μm, in particular from 14 to 30 μm. The mean particle size D₅₀ can be determined, for example, using dynamic light scattering (DLS).

The pigments (a2) represent the second essential component of the agent according to the invention and are preferably used within specific quantity ranges in the agent.

Particularly good results were obtained when the agent contained, based on the total weight of the agent, one or more pigments (a2) in a total amount of 0.01 to 10.0 wt. %, preferably 0.1 to 6.0 wt. %, more preferably 0.1 to 2.5 wt. %, and very particularly preferably 0.1 to 2.0 wt. %.

In another very particularly preferred embodiment, an agent according to the invention is characterized in that the agent contains, based on the total weight of the agent, one or more pigments (a2) in a total amount of 0.01 to 10.0 wt. %, preferably 0.1 to 6.0 wt. %, more preferably 0.1 to 2.5 wt. %, and very particularly preferably 0.1 to 2.0 wt. %.

### Amino-Functionalized Silicone Polymers (a3)

As a third ingredient essential to the invention, the agent according to the invention contains at least one amino-functionalized silicone polymer (a3). The amino-functionalized silicone polymer can alternatively also be referred to as aminosilicone or amodimethicone.

Silicone polymers are generally macromolecules with a molecular weight of at least 500 g/mol, preferably at least 1000 g/mol, more preferably at least 2500 g/mol, particularly preferably at least 5000 g/mol, and comprise repeating organic units.

The maximum molecular weight of the silicone polymer depends on the degree of polymerization (number of polymerized monomers), and the batch size, and is also determined by the polymerization method. In the context of the present invention, it is preferable that the maximum molecular weight of the silicone polymers is not more than 10⁷ g/mol, preferably not more than 10⁶ g/mol, and particularly preferably not more than 105 g/mol.

The silicone polymers comprise many Si—O repeat units, the Si atoms being able to bear organic residues such as, for example, alkyl groups or substituted alkyl groups. Alternatively, a silicone polymer is therefore also referred to as polydimethylsiloxane.

Corresponding to the high molecular weight of the silicone polymers, these are based on more than 10 Si—O repeat units, preferably more than 50 Si—O repeat units and particularly preferably more than 100 Si—O repeat units, very particularly preferably more than 500 Si—O units.

An amino-functionalized silicone polymer is understood to mean a functionalized silicone which bears at least one structural unit with an amino group. The amino-functionalized silicone polymer preferably bears a plurality of structural units with at least one amino group in each instance. An amino group is understood to mean a primary amino group, a secondary amino group and a tertiary amino group. All these amino groups can be protonated in an acidic environment and are then present in their cationic form.

In principle, it was possible to achieve positive effects of amino-functionalized silicone polymers (a3) when they bore at least one primary, at least one secondary and/or at least one tertiary amino group. However, colorings with the highest color intensities were observed when an amino-functionalized silicone polymer (a3) was used in an agent containing at least one secondary amino group.

In a very particularly preferred embodiment, an agent according to the invention is characterized in that it additionally contains
(a3) at least one amino-functionalized silicone polymer having at least one secondary amino group.

The secondary amino groups(s) can be at different positions in the amino-functionalized silicone polymer. A particularly good effect was found when an amino-functionalized silicone polymer (a3) was used that had at least one, preferably several, structural units of the formula (Si-amino).

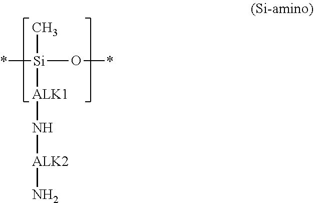

In the structural units of formula (Si-amino), the abbreviations ALK and ALK2 are each independently a linear or branched divalent C₁-C₂₀ alkylene group.

In an additional very particularly preferred embodiment, an agent according to the invention is characterized In that the agent contains at least one amino-functionalized silicone polymer (a3) which comprises at least one structural unit of formula (Si-amino),

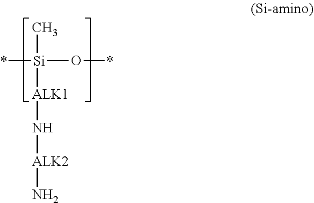

where
ALK1 and ALK2 represent, independently of one another, a linear or branched divalent C₁-C₂₀ alkylene group.

The position marked with an asterisk (*) always indicates the bond to other structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀ alkylene group are, for example, the methylene group (—CH₂—), the ethylene group (—CH₂—CH₂—), the propylene group (—CH₂—CH₂—CH₂—) and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀-alkylene groups are (—CH₂—CH(CH₃)—) and (—CH₂—CH(CH₃)—CH₂—).

In another particularly preferred embodiment, the structural units of formula (Si-amino) represent repeat units in the amino-functionalized silicone polymer (a3) so that the silicone polymer comprises multiple structural units of formula (Si-amino).

In the following, particularly well-suited amino-functionalized silicone polymers (a3) with at least one secondary amino group are listed.

Colorings with the greatest color intensities were obtainable when an agent was applied on the keratinous material which contains at least one amino-functionalized silicone polymer (a3) which comprises structural units of formula (Si-I) and formula (Si-II):

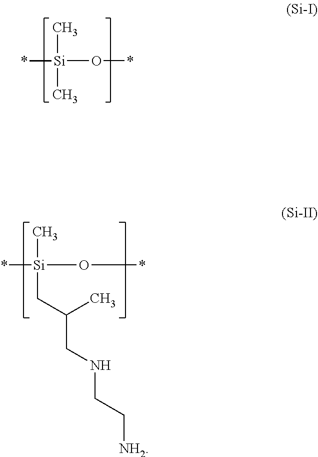

In an additional explicitly very particularly preferred embodiment, an agent according to the invention is characterized in that it contains at least one amino-functionalized silicone polymer (a3) which comprises structural units of formula (Si-I) and formula (Si-II):

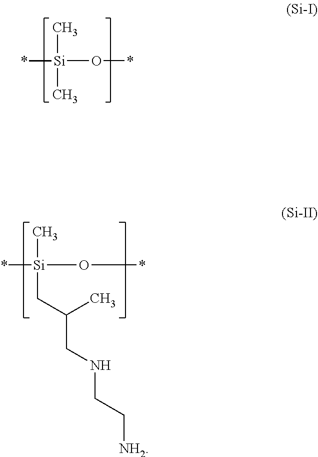

A corresponding amino-functionalized silicone polymer with the structural units (Si-I) and (Si-II) is, for example, the commercial product DC 2-8566 or Dowsil 2-8566 Amino Fluid which is sold commercially by Dow Chemical Company and which bears the designation “Siloxanes and Silicones, 3-[(2-Aminoethyl)amino]-2-methylpropyl Me, Di-Me-Siloxane” and the CAS number 106842-44-8. Another amino-functionalized silicone polymer with the structural units (Si-I) and (Si-II) is, for example, the commercial product DOWSIL™ AP-8568 Amino Fluid, which is likewise sold commercially by Dow Chemical Company.

In another preferred embodiment, an agent according to the invention is characterized in that it contains at least one amino-functional silicone polymer (a3) of the formula of the formula (Si-III):

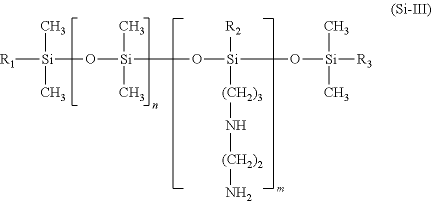

where
m and n denote numbers that are selected such that the sum (n+m) is in a range of 1 to 1000,
n is a number in a range of 0 to 999 and m is a number in a range of 1 to 1000,
R1, R2 and R3, which are identical or different, denote a hydroxyl group or a C1-4 alkoxy group,
at least one of the R1 to R3 groups denoting a hydroxyl group.

Additional agents suitable according to the invention are characterized by their content of at least one amino-functional silicone polymer (a3) of formula (Si-IV),

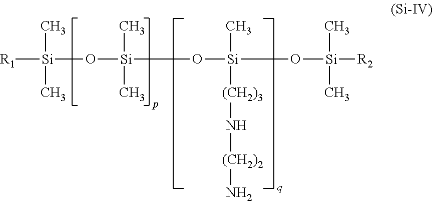

where
p and q denote numbers that are selected such that the sum (p+q) is in a range of 1 to 1000,
p is a number in a range of 0 to 999 and q is a number in a range of 1 to 1000, and
R1 and R2, which are different, denote a hydroxy group or a C1-4 alkoxy group, at least one of the groups R1 to R2 denoting a hydroxy group.

The silicones of the formulas (Si-III) and (Si-IV) differ by the grouping on the Si atom that carries the nitrogen-containing group: In formula (Si-III), R2 denotes a hydroxy group or a C1-4 alkoxy group, whereas the group in formula (Si-IV) is a methyl group. The individual Si moieties, which are labeled with the indices m and n or p and q, need not be present as blocks; instead, the individual units can also be distributed randomly, i.e., in the formulas (Si-III) and (Si-IV), each R1-Si(CH₃)₂ group is not necessarily bound to a —[O—Si(CH₃)₂] moiety.

Agents according to the invention which contain at least one amino-functional silicone polymer (a3) of formula (Si-V) have also proven to be effective with respect to the desired effects.

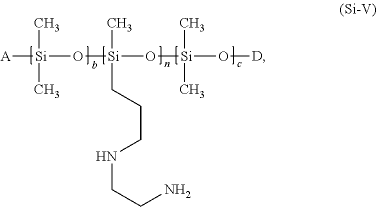

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c stand for integers between 0 and 1000,
with the proviso that

$$n > 0 and b + c > 0$$

$$at least one of the conditions A = - OH or D = - H is met .$$

In the aforementioned formula (Si-V), the individual siloxane units having the indices b, c, and n are randomly distributed, i.e., they are not necessarily block copolymers.

The agent (a) can also comprise one or more different amino-functionalized silicone polymers which are described by the formula (Si-VI):

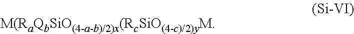

wherein, in the above formula, R is a hydrocarbon or a hydrocarbon group having 1 to approximately 6 carbon atoms, Q is a polar group of general formula —R¹HZ, in which R¹ is a bivalent, linking group bonded to hydrogen and the group Z, composed of carbon and hydrogen atoms, carbon, hydrogen and oxygen atoms, or carbon, hydrogen and nitrogen atoms, and Z is an organic, aminofunctional group containing at least one aminofunctional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)_{z}(CH₂)_{zz}NH, in which both z and zz are independently 1 or more, this structure comprising diamino ring structures, such as piperazinyl. Z is most preferably a —NHCH₂CH₂NH₂ group. Another possible formula for Z is N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-a-b)/2} units to the R_{c}SiO_{(4-c)/2} units lies within a range of about 1:2 to 1:65, preferably about 1:5 to about 1:65 and most preferably about 1:15 to about 1:20. If one or more silicones of the above formula are used, then the various variable substituents in the above formula can be different in the various silicone components, present in the silicone mixture.

Within the scope of another preferred embodiment, an agent according to the invention is characterized in that it contains at least one amino-functional silicone polymer of the formula (Si-VII):

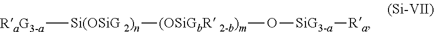

in which:
   G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
   a represents a number between 0 and 3, in particular 0;
   b represents a number between 0 and 1, in particular 1;
   m and n are numbers whose sum (m+n) is between 1 and 2000, preferably between 50 and 150, n preferably assuming values of 0 to 1999 and in particular from 49 to 149, and m preferably assuming values of 1 to 2000, in particular from 1 to 10;
   R′ is a monovalent group selected from:

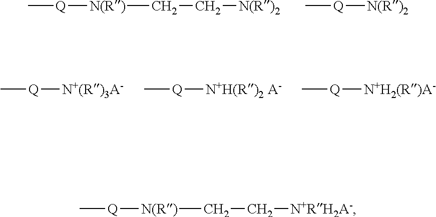

each Q representing a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—,
R″ representing identical or different functional groups from the group —H, -phenyl, -benzyl, —CH₂—CH(CH₃)Ph, from the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A representing an anion preferably selected from chloride, bromide, iodide or methosulfate.

In another preferred embodiment, an agent according to the invention is characterized in that it contains at least one amino-functional silicone polymer (a3) of the formula of the formula (Si-VIIa):

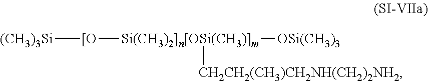

in which m and n are numbers of which the sum (m+n) is between 1 and 2000, preferably between 50 and 150, n preferably assuming values of 0 to 1999 and in particular from 49 to 149 and m preferably assuming values of 1 to 2000, in particular from 1 to 10.

These silicones are designated as trimethylsilylamodimethicones in accordance with the INCI Declaration.

In another preferred embodiment, an agent according to the invention is characterized in that it contains at least one amino-functional silicone polymer of the formula (Si-VIIb):

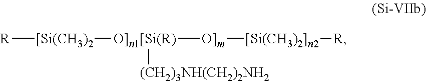

in which R denotes —OH, —O—CH₃, or a —CH₃ group and m, n1 and n2 are numbers whose sum (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10.

According to the INCI Declaration, these amino-functionalized silicone polymers are referred to as amodimethicones.

Irrespective of which amino-functional silicones are used, agents according to the invention are preferred that contain an amino-functional silicone polymer (a3) of which the amine value is above 0.25 meq/g, preferably above 0.3 meq/g, and in particular above 0.4 meq/g. The amine value here represents the milliequivalents of amine per gram of the amino-functional silicone. Said value can be determined by titration and may also be given in the unit mg KOH/g.

Furthermore, agents which contained a specific 4-morpholinomethyl-substituted silicone polymer (a3) are also suitable. This amino-functionalized silicone polymer comprises structural units of formulas (Si-VIII) and of formula (Si-IX):

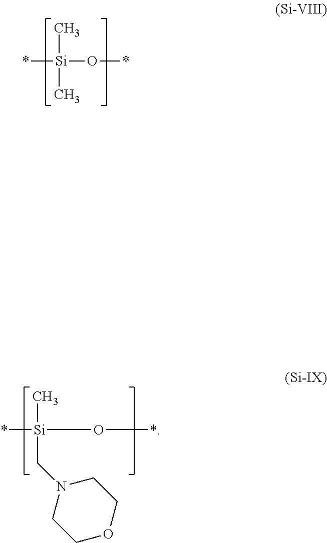

Corresponding 4-morpholinomethyl-substituted silicone polymers are described below.

A preferred amino-functionalized silicone polymer is known under the name amodimethicone/morpholinomethyl silsesquioxane copolymer and is commercially available in the form of the raw material Belsil ADM 8301 E from Wacker.

For example, a silicone which has structural units of formulas (Si-VIII), (Si-IX) and (Si-X) can be used as 4-morpholinomethyl-substituted silicone:

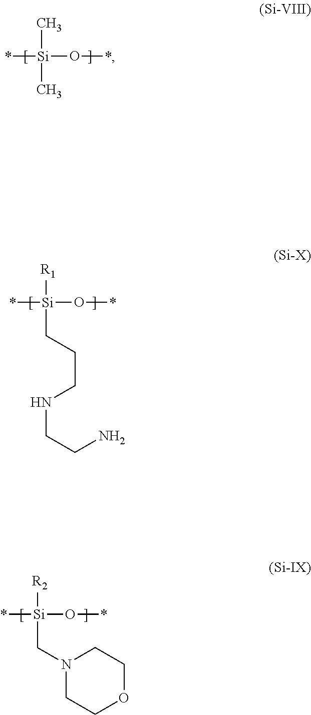

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred agents according to the invention contain at least one 4-morpholinomethyl-substituted silicone of formula (Si-XI):

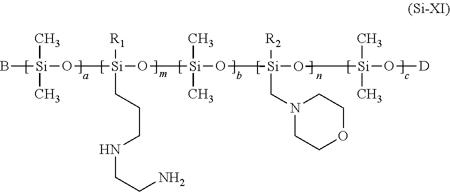

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
a, b and c represent, independently of one another, integers between 0 and 1000, with the proviso that a+b+c>0
m and n represent, independently of one another, integers between 1 and 1000,
with the proviso that
   at least one of the conditions B=—OH or D=—H is met,
   the units a, b, c, m and n are distributed randomly or in blocks in the molecule.

Structural formula (Si-XI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e., the terminal group B or D is preferably bonded to a dimethylsiloxy group. In formula (Si-VI) as well, the siloxane units a, b, c, m and n are preferably distributed randomly.

The silicones represented by formula (Si-VI) and used according to the invention can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:

| | | |
|---|---|---|
| B = —O—Si(CH₃)₂OH | and | D = —Si(CH₃)₃ |
| B = —O—Si(CH₃)₂OH | and | D = —Si(CH₃)₂OH |
| B = —O—Si(CH₃)₂OH | and | D = —Si(CH₃)₂OCH₃ |
| B = —O—Si(CH₃)₃ | and | D = —Si(CH₃)₂OH |
| B = —O—Si(CH₃)₂OCH₃ | and | D = —Si(CH₃)₂OH. |

These silicones lead to enormous improvements in the hair properties of hair treated with the agents according to the invention, and to greatly improved protection during oxidative treatment.

It has been found to be particularly advantageous if the agent according to the invention contains the amino-functionalized silicone polymer(s) (a3) in certain quantity ranges. Particularly good results were obtained when the agent contained, based on the total weight of the agent, one or more amino-functionalized silicone polymers (a3) in a total amount of 0.1 to 8.0 wt. %, preferably 0.2 to 5.0 wt. %, more preferably 0.3 to 3.0 wt. %, and very particularly preferably 0.4 to 2.5 wt. %.

Within the scope of another particularly preferred embodiment, an agent according to the invention is characterized in that it contains—based on the total weight of the agent—one or more amino-functionalized silicone polymers (a3) in a total amount of 0.1 to 8.0% by weight, preferably 0.2 to 5.0% by weight, more preferably 0.3 to 3.0% by weight and very particularly preferably 0.4 to 2.5% by weight.

### Alkylene Glycols of Formula (AG)

The agent according to the invention contains the essential components (a1), (a2), and (a3) preferably in a cosmetic carrier. This carrier can be aqueous or water-containing, for example.

However, particularly good results were obtained when the cosmetic carrier comprises at least one alkylene glycol of formula (AG).

For this reason, it is preferred if the agent according to the invention contains at least one alkylene glycol of formula (AG),

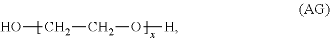

where
x is an integer of from 1 to 10,000.

Surprisingly, it has been found that the use of at least one specific alkylene glycol of formula (AG) still further improves the washing resistance of the colorings after applying the agent according to the invention to the keratin material.

The alkylene glycols of formula (AG) are protic substances having at least two hydroxyl groups which, due to their repeating —CH₂—CH₂—O— unit, can also be referred to as polyethylene glycols insofar as x is a value of at least 2. In the alkylene glycols of formula (AG), x is an integer from 1 to 10,000. In the context of the work leading to this invention, it was found that these alkylene glycols or polyethylene glycols exhibit particularly favorable suitability for, on the one hand, improving the fastness properties of the dyeing agents and, on the other hand, also optimally adjusting the viscosity of the agents.

Depending on their chain length, polyethylene glycols are liquid or solid water-soluble polymers. Polyethylene glycols with a molecular mass between 200 g/mol and 400 g/mol are non-volatile liquids at room temperature. PEG 600 has a melting range of 17 to 22° C., and therefore a pasty consistency. With molecular masses above 3000 g/mol, the PEGs are solid substances and are commercially available as flakes or powders.

Especially the use of low molecular weight alkylene glycols (or, respectively, polyethylene glycols) has proven to be well-suited for achieving the object according to the invention. In the event of low molecular weight alkylene glycols (or, respectively, polyethylene glycols) in the context of the present invention, x1 denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

In another very particularly preferred embodiment, an agent according to the invention is characterized in that it contains
(a4) at least one alkylene glycol of formula (AG-1), where

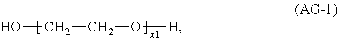

x1 is an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9 and PEG-10.

Another well-suited polyethylene glycol is PEG-32, for example. PEG-32 comprises 32 ethylene glycol units (x1=32), has a mean molar mass of 1500 g/mol and bears the CAS number 25322-68-3. PEG-32 is alternatively also referred to as PEG 1500 and can, for example, be purchased commercially from Clariant.

Furthermore, the use of high molecular weight polyethylene glycols for achieving the object according to the invention has also proven to be well suited.

High molecular weight polyethylene glycols within the meaning of the present invention can be represented by the formula (AG-2), the index number x2 standing for an integer from 101 to 10,000

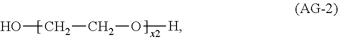

In the case of very well-suited high-molecular-weight polyethylene glycols, x2 represents an integer from 101 to 1000, preferably an integer from 105 to 800, more preferably an integer from 107 to 600, even more preferably an integer from 109 to 400 and very particularly preferably an integer from 110 to 200.

Within the scope of another very particularly preferred embodiment, an agent according to the invention is characterized in that it contains:
(a4) at least one alkylene glycol of formula (AG-2), where

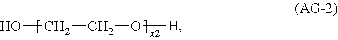

x2 denotes an integer from 101 to 1000, preferably an integer from 105 to 800, more preferably an integer from 107 to 600, even more preferably an integer from 109 to 400, and very particularly preferably an integer from 110 to 200.

A very particularly well-suited high molecular weight polyethylene glycol is, for example, PEG 6000, which can be obtained commercially from the National Starch company (China). The molecular weight of PEG 6000 is 6000 to 7500 g/mol, corresponding to an x2 value of 136 to 171.

Another well-suited polyethylene glycol is PEG 12000 which, for example, is commercially sold by CG chemicals under the trade name of polyethylene glycol 12000 S (or PEG 12000 S). The molecular weight of PEG 12000 is given at 10,500 to 15,000 g/mol, corresponding to an x2 value of 238 to 341.

Another well-suited polyethylene glycol is also PEG 20000 which is commercially available under the trade name Polyglycol 20000 P or under the alternative name PEG-350 from Clariant. For PEG 20000, an average molecular weight of 20,000 g/mol is given which corresponds to an x2 value of 454.

Surprisingly, it has been found that dyeing agents which contain both a low molecular weight polyethylene glycol and a high molecular weight polyethylene glycol have particularly favorable application properties, since these agents have both very good fastness properties and are optimized with regard to their rheological profile.

Within the scope of another explicitly very particularly preferred embodiment, an agent according to the invention is characterized in that it contains:
(a4) at least one alkylene glycol of formula (AG-1) and/or at least one alkylene glycol of formula (AG-2), where

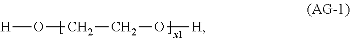

x1 represents an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and

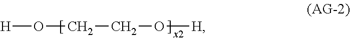

x2 denotes an integer from 101 to 1000, preferably an integer from 105 to 800, more preferably an integer from 107 to 600, even more preferably an integer from 109 to 400, and very particularly preferably an integer from 110 to 200.

To further optimize the application properties, the agent according to the invention contains the alkylene glycol(s) (AG), or (AG-1) and/or (AG-2), preferably in specific quantity ranges which, for example, based on the total weight of the agent, can be in the range of 10.0 to 99.0 wt. %, preferably 30.0 to 99.0 wt. %, more preferably 50.0 to 99.0 wt. %, and very particularly preferably 70.0 to 99.0 wt. %.

In another very particularly preferred embodiment, an agent according to the invention is characterized in that it contains, based on the total weight of the agent, one or more alkylene glycols of formula (AG-1) and/or one or more alkylene glycols of formula (AG-2) in a total amount of 10.0 to 99.0 wt. %, preferably 30.0 to 99.0 wt. %, more preferably 50.0 to 99.0 wt. % and very particularly preferably 70.0 to 99.0 wt. %.

### Water Content in the Agent

The agent described above is a ready-to-use agent which can be applied to the keratin material. This ready-to-use agent preferably possesses a medium to low water content. It has been found that particularly the agents which contain, based on the total weight of the agent, 0.1 to 70.0 wt. %, preferably 5.0 to 35.0 wt. %, more preferably 10.0 to 30.0 wt. %, and particularly preferably 12.0 to 20.0 wt. % water, are particularly well-suited.

In another explicitly very particularly preferred embodiment, an agent according to the present invention is characterized in that it contains, based on the total weight of the agent, 0.1 to 70.0 wt. %, preferably 5.0 to 35.0 wt. %, more preferably 10.0 to 30.0 wt. %, and particularly preferably 12.0 to 20.0 wt. % water.

Addition Products of C₁-C₆ Alkylene Oxide(s) to the Esters of C₁₂-C₃₀ Fatty Acids and Aromatic C₁-C₁₂ Alcohols (a5)

To further improve the fastness properties such as rub fastness and wash fastness, the agent according to the invention may additionally contain, as an optional component, at least one addition product of C₁-C₆ alkylene oxide(s) to the esters of C₁₂-C₃₀ fatty acids and aromatic C₁-C₁₂ alcohols.

The addition products of C₁-C₆ alkylene oxide(s) to the esters of C₁₂-C₃₀ fatty acids and aromatic C₁-C₁₂ alcohols (a5) are hereinafter also referred to as alkoxylated fatty acid esters (a5) for short.

C₁-C₆ alkylene oxides are the epoxides of C₁-C₆ alkanes. Examples of particularly well-suited C₁-C₆ alkylene oxides include ethylene oxide (1,2-epoxyethane), propylene oxide (1,2-epoxypropane) and butylene oxides (1,2-epoxybutane and 2,3-epoxybutane).

The ethoxylated fatty acid esters (a5) are based on C₁₂-C₃₀ fatty acids. These C₁₂-C₃₀ fatty acids according to the invention are linear or branched, saturated or mono- or polyunsaturated fatty acids which can also bear one or more hydroxyl groups. The C₁₂-C₂₄ fatty acids according to the invention are characterized in that they comprise 12 to 30 carbon atoms, preferably 12 to 24 carbon atoms. Furthermore, the C₁₂-C₂₄ fatty acids bear at least one carboxylic acid group.

To form the ethoxylated fatty acid esters (a5) according to the invention, for example one or more fatty acids can be used which are selected from the group consisting of dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], nervonic acid [(15Z)-tetracos-15-enoic acid] and/or castor oleic acid ((9Z,12R)-12-hydroxy-9-octadecenoic acid.

The ethoxylated fatty acid esters (a5) are addition products of C₁-C₆ alkylene oxide(s) to the esters of the above-described C₁₂-C₂₄ fatty acids and aromatic C₁-C₁₂ alcohols. Characteristic of aromatic C₁-C₁₂ alcohols is that they comprise 1 to 12 carbon atoms and at least one aromatic ring system. The aromatic C₁-C₁₂ alcohols each have at least one hydroxy group which can either be located directly on the aromatic compound (such as, for example, in the case of phenol), or can be linked to the aromatic compound via an aliphatic unit (such as, for example, in the case of benzyl alcohol or 2-phenoxyethanol). The structures of the aromatic C₁-C₁₂ alcohols may also comprise additional heteroatoms such as oxygen or nitrogen.

The corresponding aromatic C₁-C₁₂ alcohols may be mono- or polyvalent alcohols, i.e. the alcohols may have one or more hydroxyl groups.

Monovalent aromatic C₁-C₁₂ alcohols are very particularly preferred. This compound class comprises exactly one hydroxy group. Suitable representatives may be, for example, phenol, benzyl alcohol, 2-phenylethyl alcohol and 2-phenoxyethanol.

Within the scope of another particularly preferred embodiment, an agent according to the invention is characterized in that it contains:
(a5) at least one addition product of C₁-C₆ alkylene oxide(s) to the esters of C₁₂-C₃₀ fatty acids and aromatic C₁-C₁₂ alcohols.

Examples of particularly well-suited C₁-C₆ alkylene oxides include ethylene oxide (1,2-epoxyethane), propylene oxide (1,2-epoxypropane) and butylene oxides (1,2-epoxybutane and 2,3-epoxybutane). Ethylene oxide (1,2-epoxyethane) and propylene oxide (1,2-epoxypropane) are explicitly very particularly preferred. Propylene oxide (1,2-epoxypropane) is most preferred.

A corresponding addition product of ethylene oxide to the esters of C₁₂-C₃₀ fatty acids and aromatic C₁-C₁₂ alcohols (a5) is produced when the C₁₂-C₃₀ fatty acid itself or the ester already formed therefrom is reacted with ethylene oxide (alternative name: 1,2-epoxyethane, CAS number 75-21-8).

Analogously, a corresponding addition product of propylene oxide to the esters of C₁₂-C₃₀ fatty acids and aromatic C₁-C₁₂ alcohols is produced when the C₁₂-C₃₀ fatty acid itself or the ester already formed therefrom is reacted with propylene oxide (alternative name: 1,2-epoxypropane, CAS numbers 75-56-9 (racemate), 15448-47-2 ((R)-enantiomer, and 16088-62-3 (S)-enantiomer).

If the C₁₂-C₃₀ fatty acid itself reacts with ethylene oxide, initially an adduct can form starting from the carboxylic acid group of the C₁₂-C₃₀ fatty acid and the ethylene oxide, so that a *-C(O)—O—CH₂—CH₂—O-* group arises. This group is likewise an ester. If one mole of ethylene oxide is reacted per mole of fatty acid, a simple adduct with a *-CH2-CH2-O-* unit is formed in the agent. Depending on the molar excess used for the ethylene oxide, several adducts can also form, however, wherein there is a plurality of *-CH2-CH2-O-* units per mole C₁₂-C₃₀ fatty acid. To form the ester, this adduct is then further reacted with at least one aromatic C₁-C₁₂ alcohol. The positions marked with an asterisk represent the bond to the remainder of the fatty acid and the bond to the remaining part of the alcohol.

If the C₁₂-C₃₀ fatty acid is reacted analogously with propylene oxide, initially an adduct forms starting from the carboxylic acid group of the C₁₂-C₃₀ fatty acid and the propylene oxide, so that a *-C(O)—O—CH(CH)₃)—CH₂—O-* group or a *-C(O)—O—CH₂—CH(CH₃)—O-* group arises. A mixture of the two aforementioned moieties is usually obtained in the reaction mixture. Both moieties are likewise esters. If one mole of propylene oxide is reacted per mole of fatty acid, a simple adduct with a mixture of the units *-CH(CH₃)—CH₂—O-* and *-CH₂—CH(CH₃)—O-* is formed in the agent. Depending on the molar excess used for the propylene oxide, several adducts can also form, however, wherein there is then a plurality of *CH(CH₃)—CH₂—O-* and/or *-CH₂—CH(CH₃)—O-* units per mole C₁₂-C₃₀ fatty acid. To form the ester, this adduct is then further reacted with at least one aromatic C₁-C₁₂ alcohol. The positions marked with an asterisk represent the bond to the remainder of the fatty acid and the bond to the remaining part of the alcohol.

Furthermore, it is conceivable in principle for the C₁₂-C₃₀ fatty acid to be reacted with a mixture of ethylene oxide and propylene oxide. In this case, mixtures of the adducts described above are formed. The reactions of higher alkylene oxides, for example butylene oxides, with the C₁₂-C₃₀ fatty acids are also thereby possible.

The addition products produced in this way lead, for example, to the alkoxylated fatty acid esters of general formula (AFE-I)

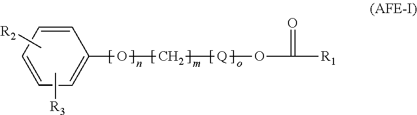

where
R7 represents a saturated or unsaturated C₁₁-C₂₉ alkyl group,
R1, R3 represent, independently of one another, a hydrogen atom, a C₁-C₆ alkyl group, a hydroxy group, or a C₁-C₆ alkoxy group.
n represents the number 0 or 1,
m represents an integer from 0 to 6,
represents an integer from 1 to 60, and
Q represents a structural unit —O—CH₂—CH₂—, —O—CH(CH₃)—CH₂— or —O—CH₂—CH(CH₃)— with the proviso that if m is equal to 0, n is also equal to 0.

With agents containing at least ethoxylated fatty acid esters (a5) of formula (AFE-I), colorings were obtained which are particularly notable with respect to favorable wash fastness and rub fastness. For this reason, the use of one or more ethoxylated fatty acid esters (a5) of formula (AFE-I) in the agents as an additional component beyond components (a1), (a2) and (a3) is very particularly preferred.

Within the scope of another particularly preferred embodiment, an agent according to the invention is characterized in that it contains:
(a5) at least one alkoxylated fatty acid ester of general formula (AFE-I)

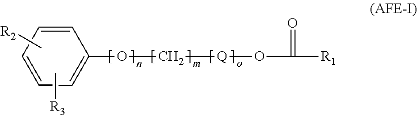

where
R1 represents a saturated or unsaturated C₁₁-C₂₉ alkyl group,
R1, R3 represent, independently of one another, a hydrogen atom, a C₁-C₆ alkyl group, a hydroxy group, or a C₁-C₆ alkoxy group.
n represents the number 0 or 1,
m represents an integer from 0 to 6,
represents an integer from 1 to 60, and
Q represents a structural unit —O—CH₂—CH₂—, —O—CH(CH₃)—CH₂— or —O—CH₂—CH(CH₃)— with the proviso that if m is equal to 0, n is also equal to 0.

The residue R1 represents a saturated or unsaturated C₁₁-C₂₉ alkyl group. An unsaturated C₁₁-C₂₃ alkyl group can comprise one or more double bonds and is alternatively also as termed an unsaturated C₁₁-C₂₃ alkenyl group. The saturated or unsaturated C₁₁-C₂₉ alkyl group may be linear or branched.

Preferably, R1 represents a linear, saturated or unsaturated C₁₁-C₂₃ alkyl group.

The residues R2 and R3 are each a hydrogen atom, a C₁-C₆ alkyl group, a hydroxy group or a C₁-C₆ alkoxy group. Very particularly preferably, the residues R1 and R2 both stand for a hydrogen atom.

The index number n is the number 0 or 1. Preferably, n is the number 0.

The index number m is an integer from 0 to 6. Preferably, m is the number 1.

The index number o is an integer from 1 to 60. Preferably, o is an integer from 1 to 30, more preferably 1 to 20, even more preferably 1 to 10, and very particularly preferably 1 to 5.

The Q residue is a structural unit —O—CH₂—CH₂—, —O—CH(CH₃)—CH₂— or —O—CH₂—CH(CH₃)—. Particularly preferably, Q is a structural unit —O—CH(CH₃)—CH₂— or —O—CH₂—CH(CH₃)—.

If o is a number greater than 1, a plurality of structural units Q are present in the compounds of formula (AFE-I) (or also of formula (AFE-II), in which case each structural unit Q can be selected independently of the other structural units Q.

Within the scope of another very particularly preferred embodiment, an agent according to the invention is characterized in that it contains:
(a5) at least one alkoxylated fatty acid ester of general formula (AFE-I)

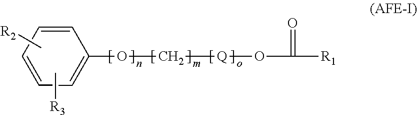

where
R7 represents a saturated or unsaturated C₁₁-C₂₉ alkyl group,
R2 and R4 are both are a hydrogen atom,
n is the number 0,
m is the number 1,
is an integer 1 to 30, preferably 1 to 20, more preferably 1 to 10, and very especially preferably 1 to 5, and
Q is a structural unit —O—CH(CH₃)—CH₂— or —O—CH₂—CH(CH₃)—.

An explicitly very particularly well-suited compound of this type is PPG-3 benzyl ether myristate, which is alternatively also referred to as α-(1-oxotetradecyl)-w-(phenylmethoxy) poly[oxy(methyl-1,2-ethanediyl)] and bears the CAS number 642443-86-5.

PPG-3 benzyl ether myristate can be purchased commercially, for example, under the trade name of Crodamol STS from Croda.

Within the scope of another very particularly preferred embodiment, an agent according to the invention is characterized in that it contains:
(a5) PPG-3 benzyl ether myristate.

The alkoxylated fatty acid esters (a5) are particularly preferably used within specific quantity ranges in the agent according to the invention. Particularly good results were obtained when the agent contained, based on the total weight of the agent, one or more alkoxylated fatty acid esters (a5) in a total amount of 0.1 to 20.0 wt. %, preferably 0.5 to 15.0 wt. %, more preferably 1.0 to 10.0 wt. %, still more preferably 1.0 to 8.0 wt. % and very particularly preferably 1.0 to 5.0 wt. %.

Addition Products of C₁-C₆ Alkylene Oxide(s) to Aliphatic C₁-C₂₄ Alkanols (a6)

To further improve the fastness properties such as rub fastness and wash fastness, the agent according to the invention may additionally contain, as an optional component, at least one addition product of C₁-C₆ alkylene oxides to C₁-C₂₄ alkanols.

The addition products of C₁-C₆ alkylene oxide(s) to aliphatic C₁-C₂₄ alkanols (a6) are also referred below as alkoxylated alkanols for short.

Within the scope of another very particularly preferred embodiment, an agent according to the invention is therefore also characterized in that it contains:
(a6) at least one addition product of C₁-C₆ alkylene oxide(s) to aliphatic C₁-C₂₄ alkanols.

The further optionally contained components (a6) can be used in addition to the ingredients of group (a5). However, the further optionally contained components (a6) can also be used instead of the ingredients of group (a5).

C₁-C₆ alkylene oxides suitable according to the invention and their preferred and particularly preferred representatives have already been defined in the preceding sections.

The C₁-C₂₄ alkanols are, in accordance with the invention, compounds having 1 to 14 carbon atoms and a hydroxyl group. The following can be mentioned by way of example: methanol, ethanol, 1-propanol, 2-propanol, 1-butanol, 2-butanol, 1-pentanol, 2-pentanol, 3-pentanol, 1-hexanol, 2-hexanol, 3-hexanol, 1-heptanol, 2-heptanol, 3-heptanol, 4-heptanol, 1-octanol, 2-octanol, 3-octanol, 4-octanol, 1-nonanol, 2-nonanol, 3-nonanol, 4-nonanol, 5-nonanol, 1-decanol, 2-decanol, 3-decanol, 4-decanol, 5-decanol, 1-undecanol, 2-undecanol, 3-undecanol, 4-undecanol, 5-undecanol, 6-undecanol, 1-dodecanol, 2-dodecanol, 3-dodecanol, 4-dodecanol, 5-dodecanol, 6-dodecanol, 1-tridecanol, 2-tridecanol, 3-tridecanol, 4-tridecanol, 5-tridecanol, 6-tridecanol, 7-tridecanol, 1-tetradecanol, 2-tetradecanol, 3-tetradecanol, 4-tetradecanol, 5-tetradecanol, 6-tetradecanol, 7-tetradecanol, 1-pentadecanol, 2-pentadecanol, 3-pentadecanol, 4-pentadecanol, 5-pentadecanol, 6-pentadecanol, 7-pentadecanol, 8-pentadecanol, 1-hexadecanol, 2-hexadecanol, 3-hexadecanol, 4-hexadecanol, 5-hexadecanol, 6-hexadecanol, 7-hexadecanol, 8-hexadecanol, 1-heptadecanol, 2-heptadecanol, 3-heptadecanol, 4-heptadecanol, 5-heptadecanol, 6-heptadecanol, 7-heptadecanol, 8-heptadecanol, 1-octadecanol, 2-octadecanol, 3-octadecanol, 4-octadecanol, 5-octadecanol, 6-octadecanol, 7-octadecanol, 8-octadecanol and 9-octadecanol.

Particularly well suited addition products of C₁-C₆ alkylene oxide(s) to aliphatic C₁-C₂₄ alkanols (a6) are the compounds of general formula (AA-I)

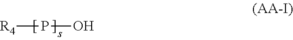

wherein
R4 represents a saturated or unsaturated C₁-C₂₄ alkyl group and
P represents a structural unit —O—CH₂—CH₂—, —O—CH(CH₃)—CH₂— or —O—CH₂—CH(CH₃)—, and
s represents an integer from 1 to 60.

The functional group R4 represents a saturated or unsaturated C₁-C₂₄ alkyl group. The functional groups R4 can be unsaturated from a carbon number of at least 2 C atoms. An unsaturated C₂-C₂₄ alkyl group can comprise one or more double bonds and is alternatively also referred to as an unsaturated C₂-C₂₄ alkenyl group. The saturated or unsaturated C₁-C₂₄ alkyl group can be linear or branched.

Particularly preferably, the functional group R4 is a saturated, unbranched C₁-C₁₂ alkyl group. Very particularly preferably, the functional group R4 is a saturated, unbranched C₁-C₆ alkyl group.

In the compounds of formula (AA-I), P represents a structural unit —O—CH₂—CH₂—, —O—CH(CH₃)—CH₂— or —O—CH₂—CH(CH₃)—. The number of structural units contained in the compounds of formula (AA-I) is obtained by the index number s. In this case, the structural units P are oriented such that the oxygen atom in each group O—CH₂—CH₂—, —O—CH(CH₃)—CH₂— and —O—CH₂—CH(CH₃)— is adjacent to the alkyl group R4, and the relevant unit —CH₂— or —CH(CH₃)— borders the hydroxy group —OH.

If s is a number greater than 1, a plurality of structural units P are present in the compounds of formula (AA-I). In this case, each structural unit P can be selected independently of the other structural units P.

The index number s represents an integer from 1 to 60, preferably an integer from 1 to 40, further preferably an integer from 10 to 30, and very particularly preferably an integer from 10 to 20.

Within the scope of another very particularly preferred embodiment, an agent according to the invention is therefore also characterized in that it contains:
(a6) at least one addition product of C₁-C₆ alkylene oxide(s) to aliphatic C₁-C₂₄ alkanols of formula (AA-I),

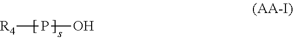

wherein
R4 represents a saturated or unsaturated C₁-C₂₄ alkyl group, preferably a C₁-C₁₂ alkyl group, particularly preferably a C₁-C₆ alkyl group, and
P represents a structural unit —O—CH₂—CH₂—, —O—CH(CH₃)—CH₂— or —O—CH₂—CH(CH₃)—, and
s represents an integer from 1 to 60, preferably an integer from 1 to 40, further preferably an integer from 10 to 30, and very particularly preferably an integer from 10 to 20.

A very particularly well-suited addition product of C₁-C₆-alkylene oxide(s) to aliphatic C₁-C₂₄ alkanols of formula (AA-I) is propylene glycol monobutyl ether which is also referred to as PPG-14 butyl ether and bears the CAS number 9003-13-8. PPG-14 butyl ether can be purchased commercially under the trade name Ucon Fluid AP from Dow.

In another very particularly preferred embodiment, an agent according to the invention is therefore further characterized in that it contains
(a6) PPG-14 butyl ether.

The alkoxylated alkanols (a6) are particularly preferably used within specific quantity ranges in the agent according to the invention.

Particularly good results were obtained when the agent contained, based on the total weight of the agent, one or more alkoxylated alkanols (a6) in a total amount of 0.1 to 20.0 wt. %, preferably 0.2 to 15.0 wt. %, more preferably 0.3 to 10.0 wt. %, still more preferably 0.4 to 5.0 wt. %, and very particularly preferably 0.5 to 3.0 wt. %.

Within the scope of another preferred embodiment, an agent according to the invention is characterized in that it contains, based on the total weight of the agent, one or more addition products of C₁-C₆ alkylene oxide(s) to aliphatic C₁-C₂₄ alkanols (a6) in a total amount of 0.1 to 20.0 wt. %, preferably 0.2 to 15.0 wt. %, more preferably 0.3 to 10.0 wt. %, still more preferably 0.4 to 5.0 wt. %, still more preferably 0.5 to 3.0 wt. %, and very particularly preferably 0.5 to 1.5 wt. %.

### Further Optional Components in the Agent

Depending on the desired form of production, the agent according to the invention can optionally also contain additional components or ingredients.

### Methods for Dyeing Keratin Materials

The agents described above can be used outstandingly in methods for dyeing keratin material, in particular human hair.

A second object of the present invention is therefore a method for dyeing keratin material, in particular human hair, in which an agent as disclosed in detail in the description of the first object of the invention is applied to the keratin fibers and, if necessary, rinsed out again after an exposure time of 30 seconds to 45 minutes.

In other words, a second object of the invention is a method for dyeing keratin material, in particular human hair, comprising the following steps:
(1) applying a dyeing agent on the keratin material, the dyeing agent being an agent as has been disclosed in detail in the description of the first object of the invention,
(2) exposing the dyeing agent on the keratin material, and
(3) rinsing out the dyeing agent with water.

In step (1) of the method according to the invention, the agent of the first object of the invention is applied to the keratin material, which is very particularly preferably human hair.

In step (2) of the method according to the invention, the agent is then allowed to act on the keratin material after its application. In this context, various exposure times of, for example, 30 seconds to 60 minutes are conceivable.

However, a great advantage of the dyeing system according to the invention is that an intensive color result can be achieved even in very short periods after short exposure times. For this reason, it is advantageous for the application mixture to remain on the keratin material after application only for comparatively short periods of 30 seconds to 15 minutes, preferably 30 seconds to 10 minutes, and particularly preferably 1 to 5 minutes.

In another preferred embodiment, a method according to the invention is characterized by
(2) exposing the keratin material to the dyeing agent for a period of 30 seconds to 15 minutes, preferably 30 seconds to 10 minutes, and more preferably 1 to 5 minutes.

After the action of the application mixture on the keratin material, said keratin material is rinsed with water in step (3) of the process.

In one embodiment, the application mixture can be washed out with water only, i.e., without the aid of an after-treatment agent or a shampoo. The use of an after-treatment agent or conditioner in step (3) is also conceivable in principle.

Method for Dyeing Keratin Material, in which the Ready-to-Use Agent is First Produced

As previously described, the agent of the first object of the invention is an agent ready for use which is either provided directly to the user in its ready-to-use form, or which is prepared by mixing various agents just prior to use.

In order to ensure a particularly fine distribution of the pigments, it has been found to be very particularly preferred to produce the ready-to-use agent shortly before application by mixing two or three different agents.

In a particularly preferred embodiment, the ready-to-use agent is accordingly prepared by mixing at least two different agents, the first of these two agents comprising the mixture of the salt(s) of divalent cations (a1) and pigment(s) (a2). Thus, the mixture of salt of the divalent cation (a1) and pigment(s) (a2) can represent, for example, a pre-dispersion which is made available in the form of a concentrate. The second agent contains at least one amino-functionalized silicone polymer (a3) and can, for example, be a water-containing cosmetic carrier formulation. To produce the ready-to-use agent, the two aforementioned agents are then shaken or stirred with each other.

Another object of the present application is therefore a method for dyeing keratin material, in particular human hair, comprising the following steps:
(1) providing an agent (I), the agent (I) containing:
(a1) at least one salt of a divalent cation, and
(a2) at least pigment,
(2) providing an agent (II), the agent (II) containing:
(a3) at least one amino-functionalized silicone polymer.
(3) producing an application mixture by mixing the agents (I) and (II),
(4) applying the application mixture produced in step (3) on the keratin material,
(5) exposing the keratin material to the application mixture applied in step (4), and
(6) rinsing out the application mixture with water,
wherein the ingredients (a1), (a2) and (a3) have already been disclosed in detail in the description of the first object of the invention.

The agent (I) in this case is preferably a predispersion of the pigments (a2) and the salt(s) of the divalent cation (a1) which can be in the form of a concentrate, for example.

The agent (II) is a base formulation which is preferably in the form of a base cream and which contains the aminosilicone(s) (a3). If the agents (I) and (II) are mixed, the predispersion of the pigments (a2) in the C₂-C₃₀ alkyl methicone (a1) ensures a particularly fine distribution of the pigments which is maintained even after the two agents (I) and (II) are mixed in the ready-to-use agent.

Depending on the selected pH, the aminosilicone (a3) in some cases has shown reduced storage stability in an aqueous medium. In these cases, it can be advantageous to also prepare the amino-functionalized silicone polymer (a3) in a separate agent and to mix both the pre-dispersion (a1)/(a2) and the aminosilicone (a3) with a base formulation only shortly before application. In this case, at least three different agents are mixed together to produce the ready-to-use dyeing agent.

The invention therefore further relates to a method for dyeing keratin material, in particular human hair, comprising the following steps:
(1) providing an agent (I), the agent (I) containing:
(a1) at least one salt of a divalent cation, and
and
(a2) at least pigment,
(2) providing an agent (II), the agent (II) being a cosmetic carrier formulation,
(3) providing an agent (III), the agent (III) containing:
(a3) at least one amino-functionalized silicone polymer (a3),
(4) preparing an application mixture by mixing agents (I) and (II) and (III),
(5) applying the application mixture prepared in step (4) to the keratin material,
(6) exposing the keratin material to the application mixture applied in step (5), and
(7) rinsing out the application mixture with water,
wherein the ingredients (a1), (a2) and (a3) have already been disclosed in detail in the description of the first object of the invention.

In this embodiment, the agent (I) is preferably a predispersion of the pigments (a2) in the salt(s) (a1), which can be in the form of a concentrate, for example. The agent (III) is preferably likewise a concentrate which contains the amino-functionalized silicone polymers (a3).

Before use, the two agents or concentrates (I) and (III) are then mixed with the carrier formulation (II). The order of mixing is arbitrary here. For example, firstly the agents (I) and (II) can be mixed together, whereupon this mixture is then mixed with the agent (III). Likewise it is conceivable to first mix the agents (II) and (III) and then to mix this mixture with the agent (I). Also, all three agents (I), (II) and (III) can be added together and then mixed first by shaking or stirring.

Finally, in the context of an additional embodiment, it can also prove advantageous to separate the components (a1) and (a2) from one another, so that the three components (a1), (a2) and (a3) which are previously separated from one another are mixed with each other during the preparation of the ready-to-use agent. This form of production can be advantageous, for example, when the pigment(s) (a2) are to be used in smaller amounts and/or in the form of a powder. The preparation of the pigments in powder form simplifies the quantitative transfer of the pigments into a mixing vessel or another container.

Another object of the present application is therefore a method for coloring keratinous material, in particular human hair, comprising the following steps:
(1) providing an agent (I), the agent (I) containing:
(a1) at least one salt of a divalent cation, and
(2) providing an agent (II), the agent (II) containing:
(a2) at least pigment,
(3) providing an agent (III), the agent (III) containing:
(a3) at least one amino-functionalized silicone polymer,
(4) preparing an application mixture by mixing agents (I) and (II) and (III),
(5) applying the application mixture prepared in step (4) to the keratin material,
(6) exposing the keratin material to the application mixture applied in step (5), and
(7) rinsing out the application mixture with water,
wherein the ingredients (a1), (a2) and (a3) have already been disclosed in detail in the description of the first object of the invention.

In relation to the additional preferred embodiments of the method according to the invention, what has been said about the agent according to the invention applies mutatis mutandis.

### Examples

### 1. Formulations

The following ready-to-use dyeing agents were prepared (all data are in percent by weight unless stated otherwise):

| | without | |
|---|---|---|
| | water | 10% CaCl2 |
| | Comparison | Invention |
| | C1 (wt. %) | I1 (wt. %) |
| PPG-14 butyl ether | 1.00 | 1.00 |
| PPG-3 benzyl ether myristate | 1.50 | 1.50 |
| Blue pigment (Goldmann Pigment Blue 60) | 0.18 | 0.18 |
| Silver pigment (Alegrace Marvelous D | 0.25 | 0.25 |
| 12/77-1 Shiny Silver, 10%) | | |
| Amino-functionalized silicone polymer | 0.75 | 0.75 |
| DOWSIL AP-8568 Amino Fluid | | |
| Water dist. | — | 10.00 |
| Calcium chloride (CaCl₂), hexahydrate | — | 10.00 |
| Sodium chloride | — | — |
| Polyethylene glycol 6000 | 15.00 | 15.00 |
| Polyethylene glycol 400 | up to 100 | up to 100 |

| | 10% water | 10% NaCl |
|---|---|---|
| | Comparison | Comparison |
| | C2 (wt. %) | C3 (wt. %) |
| PPG-14 butyl ether | 1.00 | 1.00 |
| PPG-3 benzyl ether myristate | 1.50 | 1.50 |
| Blue pigment (Goldmann Pigment Blue 60) | 0.18 | 0.18 |
| Silver pigment (Alegrace Marvelous | 0.25 | 0.25 |
| D 12/77-1 Shiny Silver, 10%) | | |
| Amino-functionalized silicone polymer | 0.75 | 0.75 |
| DOWSIL AP-8568 Amino Fluid | | |
| Water dist. | 10.00 | 10.00 |
| Calcium chloride (CaCl2) hexahydrate | — | — |
| Sodium chloride | — | 10.00 |
| Polyethylene glycol 6000 | 15.00 | 15.00 |
| Polyethylene glycol 400 | up to 100 | up to 100 |

### 2. Application

The ready-to-use dyeing agents C₁, I₁, C₂ and C₃ produced beforehand were each applied to hair strands (Kerling, type “European-hair white” (ENH)) (liquor ratio: 1 g agent per g of hair strand) and allowed to act for three minutes. Subsequently, the hair strands were washed thoroughly (1 minute) with water and dried.

### 3. Measurement of Wash Fastness

After drying, the colored strands were measured using a colorimeter from Datacolor, type Spectraflash 450.

Each strand was then washed three times (or six times) manually. To this end, each strand was moistened with water, then a commercial shampoo (Schauma 7-Kräuter) was applied to the strand (0.25 g shampoo per 1 g of hair) and massaged with the fingers 30 seconds. The strand was then rinsed for 1 minute under running lukewarm water, and the strand of hair was dried. The process described above corresponds to hair washing. For each additional hair wash, the process was repeated. The strands were colorimetrically measured again after 3 hair washes and after 6 hair washes.

The dE value used for the assessment of wash fastness is derived from the L*a*b* colorimetric values measured from the respective strand as follows:

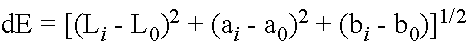

L₀, a₀ ad b₀=measured values before dyeing
Lᵢ, aᵢ and bᵢ=measured values directly after dyeing, after 3 hair washes and after 6 hair washes

The smaller the dE value, the lower the color distance between the undyed and dyed hair, and the lower the intensity of the dyed hair.

| | | dE (uncolored | dE (uncolored |
|---|---|---|---|
| | dE (uncolored | versus 3 | versus 6 |
| | versus colored) | hair washes) | hair washes) |
| C1 (without water) | 25.97 | 13.92 | 12.04 |
| I1 (10 wt. % CaCl2) | 30.05 | 25.80 | 18.11 |
| C2 (10 wt. % water) | 28.86 | 16.52 | 7.72 |
| C3 (10 wt. % NaCl) | 31.42 | 18.51 | 14.71 |

dE (uncolored versus colored) indicates the color distance between the undyed and dyed hair. The lower the dE value, the poorer the color intensity. Here, I₁ (with 10% calcium chloride (hexahydrate)) exhibits a minimally poorer color uptake compared with C3 (with 10 wt. % sodium chloride).

dE (uncolored vs 3 hair washes) indicates the color distance between the undyed and dyed hair and then the hair washed three times. The lower this dE value, the poorer the color intensity after 3 hair washes. Here, I₁ exhibits the highest color intensity, i.e., after 3 hair washes the hair strands dyed with I₁ still have a much higher color intensity. The strands dyed with I₁ therefore have the best wash fastness, which is greatly improved compared with C3.

dE (uncolored vs 6 hair washes) indicates the color distance between the undyed and dyed hair and then the hair washed six times. The lower this dE value, the poorer the color intensity after 6 hair washes. Here, I₁ exhibits the highest color intensity, i.e., after 6 hair washes the hair strands dyed with I₁ still have the highest color intensity. The strands dyed with I₁ have the best wash fastness, which is greatly improved compared with C3.

1. An agent for dyeing keratin material, in particular human hair, containing:
(a1) at least one salt of a divalent cation, and
(a2) at least one pigment, and
(a3) at least one amino-functionalized silicone polymer.

2. The agent according to claim 1, characterized in that it contains:
(a1) at least one salt of a divalent calcium cation and/or magnesium cation, which is preferably selected from the group consisting of calcium chloride, magnesium chloride, calcium bromide, magnesium bromide, calcium hydroxide, magnesium hydroxide, calcium citrate, magnesium citrate, calcium salicylate, magnesium salicylate, calcium phosphate, magnesium phosphate, calcium sulfate, magnesium sulfate, calcium tartrate, magnesium tartrate, calcium lactate, magnesium lactate, calcium malate, magnesium malate, calcium succinate, magnesium succinate, calcium benzoate, magnesium benzoate, calcium fumarate, magnesium fumarate, calcium maleinate, magnesium maleinate, calcium acetate and magnesium acetate.

3. The agent according to claim 1, characterized in that it contains, based on the total weight of the agent, one or more salts of divalent cations (a1) in a total amount from 0.1 to 25 wt. %, preferably 0.5 to 20 wt. %, more preferably from 1.0 to 15 wt. %, still more preferably from 1.5 to 10 wt. %, and very particularly preferably from 2.5 to 7.5 wt. %.

4. The agent according to claim 1, characterized in that it contains at least one inorganic pigment (a2), which is selected from the group of colored metal oxides, metal hydroxides, metal oxide hydrates, silicates, metal sulfides, complex metal cyanides, metal sulfates, bronze pigments and/or from mica-based colored pigments which are coated with at least one metal oxide and/or a metal oxychloride.

5. The agent according to claim 1, characterized in that it contains at least one pigment (a2) from the group of platelet-type metal pigments, which comprises at least one metal from the group of aluminum, copper, silver, gold, platinum, zinc, chromium, molybdenum and iron, particularly preferably aluminum.

6. The agent according to claim 1, characterized in that it contains at least one organic pigment (a2), which is preferably selected from the group consisting of carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570 or CI 74260, orange pigments with the Color Index numbers CI 11725, CI 15510, CI 45370, CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

7. The agent according to claim 1, characterized in that it contains, based on the total weight of the agent, one or more pigments (a2) in a total amount from 0.01 to 10.0 wt. %, preferably 0.1 to 6.0 wt. %, more preferably from 0.1 to 2.5 wt. %, and very particularly preferably from 0.1 to 2.0 wt. %.

8. The agent according to claim 1, characterized in that it contains
(a3) at least one amino-functionalized silicone polymer having at least one secondary amino group.

9. The agent according to claim 1, characterized in that it comprises at least one amino-functionalized silicone polymer (a3) comprising at least one structural unit of formula (Si-amino), 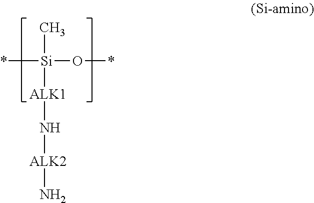
where
ALK1 and ALK2 represent, independently of one another, a linear or branched divalent C₁-C₂₀ alkylene group.

10. The agent according to claim 1, characterized in that it contains at least one amino-functionalized silicone polymer (a3) comprising structural units of formula (Si-I) and formula (Si-II)

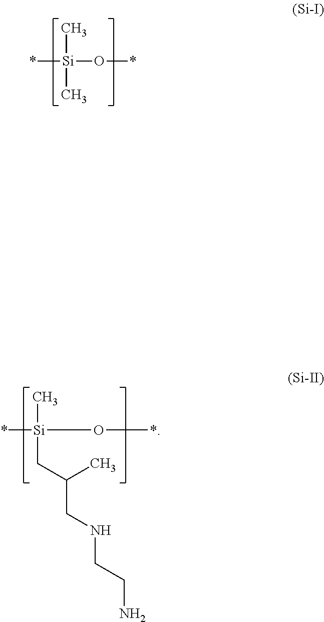

11. The agent according to claim 1, characterized in that it contains, based on the total weight of the composition, one or more amino-functionalized silicone polymers (a3) in a total amount from 0.1 to 8.0 wt. %, preferably from 0.2 to 5.0 wt. %, more preferably from 0.3 to 3.0 wt. %, and very particularly preferably from 0.4 to 2.5 wt. %.

12. The agent according to claim 1, characterized in that it contains
(a4) at least one alkylene glycol of formula (AG-1) and/or at least one alkylene glycol of formula (AG-2), where 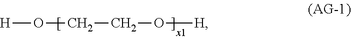
x1 represents an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and 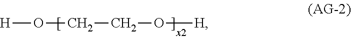
x2 denotes an integer from 101 to 1000, preferably an integer from 105 to 800, more preferably an integer from 107 to 600, even more preferably an integer from 109 to 400, and very particularly preferably an integer from 110 to 200.

13. The agent according to claim 1, characterized in that it contains, based on the total weight of the agent, one or more alkylene glycols of formula (AG-1) and/or one or more alkylene glycols of formula (AG-2) in a total amount from 10.0 to 99.0 wt. %, preferably from 30.0 to 99.0 wt. %, more preferably from 50.0 to 99.0 wt. %, and very particularly preferably from 70.0 to 99.0 wt. %.

14. The agent according to claim 1, characterized in that it contains, based on the total weight of the agent, from 0.1 to 70.0 wt. %, preferably from 5.0 to 35.0 wt. %, more preferably from 10.0 to 30.0 wt. %, and particularly preferably from 12.0 to 20.0 wt. %, water.

15. The agent according to one of claims 1 to 14, characterized in that it contains
(a5) at least one alkoxylated fatty acid ester of general formula (AFE-I) 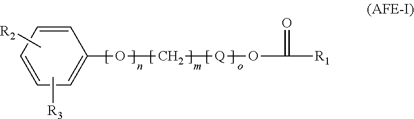
where
R1 represents a saturated or unsaturated C₁₁-C₂₉ alkyl group,
R1, R3 represent, independently of one another, a hydrogen atom, a C₁-C₆ alkyl group, a hydroxy group, or a C₁-C₆ alkoxy group.
n represents the number 0 or 1,
m represents an integer from 0 to 6,
o represents an integer from 1 to 60, and
Q represents a structural unit —O—CH₂—CH₂—, —O—CH(CH₃)—CH₂— or —O—CH₂—CH(CH₃)— with the proviso that if m is equal to 0, n is also equal to 0.

16. The agent according to claim 1, characterized in that it contains
(a6) at least one addition product of C₁-C₆ alkylene oxide(s) to aliphatic C₁-C₂₄ alkanols of formula (AA-I), 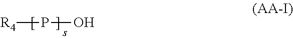
wherein
R4 represents a saturated or unsaturated C₁-C₂₄ alkyl group, preferably a C₁-C₁₂ alkyl group, particularly preferably a C₁-C₆ alkyl group, and
P represents a structural unit —O—CH₂—CH₂—, —O—CH(CH₃)—CH₂— or —O—CH₂—CH(CH₃)—, and
s represents an integer from 1 to 60, preferably an integer from 1 to 40, further preferably an integer from 10 to 30, and very particularly preferably an integer from 10 to 20.

17. A method for dyeing keratin material, in particular human hair, wherein an agent as described in claim 1 is applied to the keratin material and optionally rinsed out again after an exposure time of 30 seconds to 45 minutes.
